(19) United States
(12) Patent Application Publication
(10) Pub. No.: US 2022/0280558 A1
(43) Pub. Date: Sep. 8, 2022

(54) MOLECULAR HYDROGEN-CONTAINING COMPOSITION FOR PREVENTING OR IMPROVING OSTEOPOROSIS

(71) Applicant: MiZ Company Limited, Kamakura-shi (JP)

(72) Inventors: Fumitake SATOH, Kanagawa (JP); Yusuke ICHIKAWA, Kanagawa (JP); Bunpei SATO, Kanagawa (JP); Shinichi HIRANO, Kanagawa (JP); Hatuko NAGASAKI, Kanagawa (JP)

(21) Appl. No.: 17/686,135

(22) Filed: Mar. 3, 2022

(30) Foreign Application Priority Data
Mar. 5, 2021 (JP) 2021-072182

(51) Int. Cl. A61K 33/00; A61K 9/00; A61P 19/10

(52) U.S. Cl. CPC A61K 33/00; A61K 9/007; A61P 19/10

(57) ABSTRACT

The present invention provides a composition for preventing or improving human osteoporosis, comprising molecular hydrogen as an active ingredient. More specifically, the present invention provides a composition for preventing or improving human osteoporosis, wherein the human osteoporosis is primary osteoporosis and/or secondary osteoporosis.

### RELATED APPLICATIONS

This application claims priority to Japanese Patent Application No. 2021-072182, filed on Mar. 5, 2021, the entire content of which is incorporated herein by reference.

### BACKGROUND OF THE INVENTION

### 1. Field of the Invention

The present invention provides a molecular hydrogen-containing composition for preventing or improving osteoporosis.

### 2. Description of the Related Art

Human bone has a porous structure, and its strength is maintained by a mesh-like bone crosslink. The bone crosslink maintains a constant state by destroying old bone while forming new bone, through bone formation by osteoblasts and bone resorption by osteoclasts. The clinical condition of osteoporosis is characterized by fragility of a bone crosslink due to relatively increased bone resorption. Since the relatively increased bone resorption is accelerated by a decrease in sex hormone levels, osteoporosis is common among postmenopausal women. Further, since women inherently have lower bone mass than men, symptoms are easily uncovered when a balance of formation and resorption is lost. Femur or vertebral body fracture may make elderly people so-called bedridden, markedly lowering their quality of life.

Hydrogen, the active ingredient of the present invention, can eliminate hydroxyl radicals having the highest oxidation activity among such reactive oxygens, and convert those into water molecules (Fukai Yuh, “Suiso Bunshi wa Kanari Sugoi”, Kobunsha Co., Ltd., 2017, p. 24). Because hydrogen acts as an antioxidant which eliminates hydroxyl radicals, there are many reports that administration of hydrogen can cure diseases associated with oxidative stress. Reports on osteoporosis include a report showing that loss of bone density could be suppressed in ovariectomized mice by administering hydrogen water thereto for three months (Ji-Dong Guo, et al., “Hydrogen water consumption prevents osteopenia in ovariectomized rats.” British Journal of Pharmacology, 2013; 168:1412-1420). However, small animals such as disease model mice and human are different species and thus are different in not only the appearance but also DNA. Thus, the improving effects of hydrogen demonstrated in animal tests are not always guaranteed in human (Fukai Yuh, op. cit., p. 57).

The medical treatment effects of hydrogen significantly depend on the administration method, namely, inhalation of hydrogen gas or drinking of hydrogen water. Accordingly, selection of the administration method depending on the symptoms of diseases should be examined in detail as one of problems of medical treatments with hydrogen in the future (Fukai Yuh, op. cit., p. 92).

Furthermore, because high concentration hydrogen molecules are explosive, hydrogen having a low concentration equal to or less than the explosion limit should be used in treatments and maintenance. However, it should also be verified whether hydrogen having a low concentration equal to or less than the explosion limit demonstrates sufficient maintenance and improving effects against each of the diseases.

Although there are many reports about prevention or effects against diseases in animals including humans, such effects in these reports are all derived from use of a high concentration (66% or 67%) hydrogen gas generator, and risks of explosion remain still.

### SUMMARY OF THE INVENTION

To solve the above problems, the present invention reports, for the first time ever, that osteoporosis was prevented or improved with a low hydrogen concentration equal to or less than the explosion limit (e.g., equal to or less than the detonation limit of 18.5%).

That is, the present invention encompasses the following characteristics:

(1) A composition for preventing or improving human osteoporosis, comprising molecular hydrogen as an active ingredient.

(2) The composition according to (1), wherein the human osteoporosis is primary osteoporosis and/or secondary osteoporosis.

(3) The composition according to (1) or (2), wherein the preventing or improving human osteoporosis is maintaining or raising bone density.

(4) The composition according to any of (1) to (3), wherein a gas comprising the molecular hydrogen has a hydrogen concentration of higher than zero (0) and not higher than 18.5% by volume.

(5) The composition according to any of (1) to (4), wherein the composition comprising the molecular hydrogen as the active ingredient is fed to a subject by inhalation of the composition using a hydrogen gas supply apparatus.

Prevention or improvement of human osteoporosis can be improved even with a low hydrogen concentration equal to or less than the explosion limit.

### Advantageous Effects of Invention

The present invention provides a composition and/or a method for preventing or improving human osteoporosis, the composition and/or the method comprising molecular hydrogen as an active ingredient.

### DETAILED DESCRIPTION OF THE PREFERRED EMBODIMENTS

The present invention will be described in more detail below.

### 1. Composition or Method for Preventing or Improving Human Osteoporosis

The present invention provides a composition for preventing or improving human osteoporosis, comprising molecular hydrogen as an active ingredient.

In the present specification, the term “human osteoporosis” refers to a disease in which bone strength is decreased by acquired loss of bone density or deterioration of bone quality, leading to easy fracture, or such a condition, and/or a disease associated therewith.

In the present specification, the term “human osteoporosis” includes primary osteoporosis associated with aging and secondary osteoporosis caused by diseases, drugs such as, pharmaceutical products including in particular, steroids, nutrition, or the like.

In the present specification, whether or not “osteoporosis” has occurred can be determined according to bone density criteria. Usually, osteoporosis is diagnosed with bone density of 70% or lower, and osteopenia with a high risk of osteoporosis is diagnosed with bone density of 70% to 80%.

In the present specification, bone density can be measured by an existing measuring method, for example, by dual-energy-x-ray absorptiometry (DXA) of the trunk bone.

In the present specification, the term “subject” includes mammalians such as primates including humans, pet animals such as dogs and cats, and ornamental animals such as zoo animals. Preferred subjects are humans.

The term “human” includes a patient having a disease included in the present invention. However, the term “human” does not include disease model animals used in animal tests, such as mice, rats, dogs, cats, monkeys, pigs, fish, and insects. The disease model animals have diseases basically different from those of patients (including mammalians (pet animals) diagnosed by veterinarians) diagnosed by medical practitioners, such as medical doctors and veterinarians, in clinical sites such as medical institutions.

In the present specification, “hydrogen,” the active ingredient of the composition of the present invention, is molecular hydrogen (i.e., gaseous hydrogen or hydrogen gas) and is simply referred to as “hydrogen” or “hydrogen gas” unless otherwise specified. Additionally, the term “hydrogen” used in the present specification refers to a molecular formula of H₂, D₂ (deuterium), or HD (deuterated hydrogen) or a gas mixture thereof. D₂ is expensive but known to have a stronger superoxide eliminating effect than that of H₂. Hydrogen that can be used in the present invention is H₂, D₂ (deuterium), HD (deuterated hydrogen), or a gas mixture thereof, preferably H₂. Alternatively, D₂, and/or HD can be used instead of H₂ or in a mixture with H₂.

Preferred embodiments of the composition of the present invention are gases or liquids containing molecular hydrogen, preferably gases containing molecular hydrogen.

The gases containing molecular hydrogen are preferably air containing hydrogen gas or a mixed gas containing hydrogen gas and oxygen gas. The concentration of hydrogen gas in a gas containing molecular hydrogen is higher than zero (0) and not higher than 18.5% by volume, for example, 0.5% to 18.5% by volume, preferably 1% to 10% by volume, for example, 2% to 8% by volume, 2% to 9% by volume, 2% to 10% by volume, 3% to 6% by volume, 3% to 7% by volume, 3% to 8% by volume, 3% to 9% by volume, 3% to 10% by volume, 4% to 5% by volume, 4% to 6% by volume, 4% to 7% by volume, 4% to 8% by volume, 4% to 9% by volume, 4% to 10% by volume, 5% to 8% by volume, 5% to 9% by volume, 5% to 10% by volume, 6% to 7% by volume, 6% to 8% by volume, 6% to 9% by volume, 6% to 10% by volume, and the like. In the present invention, higher hydrogen gas concentration (but equal to or less than the explosion limit) or larger daily hydrogen doses tend to be associated with greater effects of maintaining or improving the disease of the human or the animal. In the present invention, a hydrogen gas supply apparatus which generates and emits hydrogen in the safety concentration above should be used.

Because hydrogen is a gas without any accompanied pharmacological side effects, the inhalation time is not limited. The action mechanism of hydrogen is to eliminate hydroxyl radicals generated inside cells, and particularly inside mitochondria to protect cells and substances forming cells from oxidative stress or oxidation. Accordingly, the estimated inhalation amount of hydrogen needed to eliminate hydroxyl radicals and peroxynitrite to protect cells from oxidative stress can be calculated from the number of cells forming a body or the number of mitochondria inside cells. To be noted, a larger amount of hydrogen should be inhaled to sufficiently feed hydrogen to the inside of a relatively large organism such as a liver.

From such a viewpoint, the inhalation time of hydrogen is preferably within 1 hour daily. More preferably, the daily inhalation time is preferably any time selected from the range of 6 hours or less, such as 2 hours or less, 3 hours or less, 4 hours or less, and 5 hours or less. Still more preferably, the daily inhalation time is preferably any time selected from the range of 12 hours or less, such as 7 hours or less, 8 hours or less, 9 hours or less, 10 hours or less, and 11 hours or less. Further still more preferably, the daily inhalation time is preferably any time selected from the range of 24 hours or less, such as 13 hours or less, 14 hours or less, 15 hours or less, 16 hours or less, 17 hours or less, 18 hours or less, 19 hours or less, 20 hours or less, 21 hours or less, 22 hours or less, and 23 hours or less.

Hydrogen is a flammable and explosive gas, and use of a hydrogen gas having a hydrogen concentration of 18.5% or more should be avoided because it involves not only explosion but also detonation. Accordingly, it is preferable to add hydrogen to the composition of the present invention under conditions safe for subjects such as humans and administer the mixture to subjects to prevent or improve human osteoporosis.

When a gas other than hydrogen gas is air, the air concentration is in the range of, for example, 81.5% to 99.5% by volume. When a gas other than hydrogen gas is a gas containing oxygen gas, the oxygen gas concentration is in the rage of, for example, 21% to 99.5% by volume. As another main gas, for example, nitrogen gas can be further added.

The liquids containing molecular hydrogen are specifically aqueous liquids containing a dissolved hydrogen gas. Examples of the aqueous liquids used herein include, but are not limited to, water (e.g., purified water, sterilized water), physiological saline, buffer solutions (e.g., buffer solutions of pH 4 to 7.4), drip infusion solutions, fluid infusion solutions, injection solutions, and drinks (e.g., tea drinks such as green tea and black tea, fruit juice, green juice, vegetable juice). Examples of the hydrogen concentration in a liquid containing molecular hydrogen include, but are not limited to, 1 to 10 ppm, preferably 1.2 to 9 ppm, for example, 1.5 to 9 ppm, 1.5 to 8 ppm, 1.5 to 7 ppm, 1.5 to 6 ppm, 1.5 to 5 ppm, 1.5 to 4 ppm, 2 to 10 ppm, 2 to 9 ppm, 2 to 8 ppm, 2 to 7 ppm, 2 to 6 ppm, 2 to 5 ppm, 3 to 10 ppm, 3 to 9 ppm, 3 to 8 ppm, 3 to 7 ppm, 4 to 10 ppm, 4 to 9 ppm, 4 to 8 ppm, 4 to 7 ppm, 5 to 10 ppm, 5 to 9 ppm, 5 to 8 ppm, and 5 to 7 ppm.

A gas or a liquid containing molecular hydrogen is formulated to provide a predetermined hydrogen gas concentration and then with the same, for example, a pressure-resistant container (e.g., a stainless cylinder, an aluminum can, a pressure-resistant plastic bottle [e.g., a pressure-resistant PET bottle] and a plastic bag preferably having the inside laminated with an aluminum film, or an aluminum bag) is filled. Aluminum has the property of unlikely allowing hydrogen molecules to pass therethrough. Alternatively, a gas containing molecular hydrogen or a liquid containing molecular hydrogen may be produced in situ before use by using an apparatus such as a hydrogen gas generating apparatus, a hydrogen water generating apparatus, or a hydrogen gas adding apparatus such as a known or commercially available hydrogen gas supply apparatus (an apparatus for generating a gas containing molecular hydrogen), a hydrogen adding device (an apparatus for hydrogen water generation), or a non-destructive hydrogen adding apparatus (e.g., an apparatus for non-destructively adding hydrogen gas into a bag for a biocompatible solution such as a drip infusion solution).

The hydrogen gas supply apparatus enables hydrogen gas generated from a reaction of a hydrogen generating agent (e.g., metallic aluminum, magnesium hydride) and water to be mixed with a diluent gas (e.g., air, oxygen) in a predetermined ratio (refer to Japanese Patent No. 5228142, etc.). Or, the hydrogen gas supply apparatus mixes hydrogen gas generated utilizing electrolysis of water with a diluent gas such as oxygen or air (refer to Japanese Patent No. 5502973, Japanese Patent No. 5900688, etc.). Thus, a gas containing molecular hydrogen at a hydrogen concentration in the range of, for example, 0.5% to 18.5% by volume can be prepared.

The hydrogen adding device is an apparatus that generates hydrogen by using a hydrogen generating agent and a pH modifier and dissolving the hydrogen in a biocompatible solution such as water (refer to Japanese Patent No. 4756102, Japanese Patent No. 4652479, Japanese Patent No. 4950352, Japanese Patent No. 6159462, Japanese Patent No. 6170605, Japanese Patent Laid-open No. 2017-104842, etc.). Examples of a mixture of a hydrogen generating agent and a pH modifier include metallic magnesium and a strongly acidic ion exchange resin or an organic acid (e.g., malic acid, citric acid) and a metallic aluminum powder and a calcium hydroxide powder. With these mixtures, a liquid containing molecular hydrogen at a dissolved hydrogen concentration of, for example, approximately 1 to 10 ppm can be prepared.

The non-destructive hydrogen adding apparatus is an apparatus or a device that adds hydrogen gas to a commercially available biocompatible solution such as a drip infusion solution (e.g., enclosed in a hydrogen-permeable plastic bag such as a polyethylene bag) from the outside of a package and is commercially available from, for example, MiZ Company Limited (http://www.e-miz.co.jp/echnology.html). This apparatus can dissolve hydrogen in a biocompatible solution aseptically until the equilibrium concentration is reached, by immersing a bag containing the biocompatible solution in saturated hydrogen water, so that hydrogen is permeated into the bag. The apparatus is composed of, for example, an electrolytic bath and a water bath, and water in the water bath is circulated in the electrolytic bath and the water bath to generate hydrogen by electrolysis. Or, a simplified, disposable device can be used for a similar purpose (refer to Japanese Patent Laid-open No. 2016-112562, etc.). This device has a biocompatible solution-containing plastic bag (a hydrogen-permeable bag, for example, a polyethylene bag) and a hydrogen generating agent (e.g., metallic calcium, metallic magnesium/cation exchange resin) incorporated in an aluminum bag, and the hydrogen generating agent is wrapped with, for example, a non-woven fabric (e.g., steam-permeable non-woven fabric). Hydrogen generated by wetting the hydrogen generating agent wrapped with a non-woven fabric with a small amount of water, such as a steam, is dissolved in a biocompatible solution non-destructively and aseptically.

Or, a purified hydrogen gas cylinder, a purified oxygen gas cylinder, or a purified air cylinder may be provided to produce a gas or a liquid containing molecular hydrogen which is adjusted to provide a predetermined hydrogen concentration or a predetermined oxygen or air concentration.

The gas containing molecular hydrogen or the liquid (such as water (such as purified water and sterilized water), physiological saline, or drop infusion solutions) containing molecular hydrogen prepared using the above-mentioned apparatuses or the devices can be administered orally or parenterally to subjects with osteoporosis before, during, or after surgery.

Other embodiments of the composition of the present invention include dosage forms (e.g., tablets, capsules) prepared to be orally administered to (or ingested by) subjects, which contain a hydrogen generating agent that enables hydrogen to be generated in the gastrointestinal tract. The hydrogen generating agent preferably comprises, for example, components approved as food or food additives.

When the composition of the present invention comprises molecular hydrogen as an active ingredient, examples of the method of administering the composition to subjects include administration by inhalation, suction or the like. For example, transpulmonary administration is preferred. When a liquid containing molecular hydrogen is contained as an active ingredient, oral or intravenous administration (including drip infusion) is preferred. When a gas is inhaled, the gas can be inhaled from the mouth or the nose to the lung via a nasale cannula, a mask-like device covering the mouth and the nose, or a hydrogen feedable chamber such as a chamber, and then can be delivered to the whole body by blood.

The liquid containing molecular hydrogen to be orally administered may be administered to subjects as a cooled liquid or a liquid stored at room temperature. Hydrogen is dissolved in water at a concentration of approximately 1.6 ppm (1.6 mg/L) at room temperature and under a normal pressure, and the difference in solubility due to temperature is known to be relatively small. Or, when a liquid containing molecular hydrogen is, for example, in the form of a drip infusion solution or an injection solution containing hydrogen gas prepared using the above-described non-destructive hydrogen adding apparatus, the liquid may be administered to subjects by parenteral routes, such as intravenous or intraarterial administration.

One dose or multiple doses (e.g., two to three doses) per day of a gas containing molecular hydrogen at the above-mentioned hydrogen concentrations or a liquid containing molecular hydrogen at the above-mentioned dissolved hydrogen concentrations can be administered to humans for a period of one week to three months or longer, for example, one week to six months or longer (e.g., one year or longer, two years or longer). When a gas containing molecular hydrogen is administered, the gas is preferably inhaled for at least 30 minutes per dose. Because the improving effect becomes higher with a longer inhalation time, the gas can be administered for, for example, 30 minutes to one hour, one hour to two hours, two hours to three hours, or longer. Additionally, when a gas containing molecular hydrogen is administered in a transpulmonary manner by inhalation or suction, the gas can be administered to subjects under an atmospheric pressure environment, or, for example, under a high atmospheric pressure in the range exceeding a standard atmospheric pressure (i.e., approximately 1.013 atm) and not higher than 7.0 atm, for example, under a high atmospheric pressure environment in the range of 1.02 to 7.0 atm, preferably in the range of 1.02 to 5.0 atm, more preferably in the range of 1.02 to 4.0 atm, yet more preferably in the range of 1.02 to 1.35 atm (including the gas containing molecular hydrogen).

### 2. Method for Preventing or Improving Human Osteoporosis

The present invention further provides a method for accelerating recovery or improvement of a subject with osteoporosis from invasion in surgery and/or a symptom associated with the surgery using the composition comprising molecular hydrogen as the active ingredient.

The composition comprising molecular hydrogen, symptoms associated with human osteoporosis, the dose, the administration method, and the like are as described in the above 1.

In the method of the present invention, a gas containing molecular hydrogen (preferably, air or oxygen) at higher than zero (0) and not higher than 18.5% by volume, for example, 0.5% to 18.5% by volume, 2% to 10% by volume, 2% to 9% by volume, 2% to 8% by volume, 3% to 10% by volume, 3% to 9% by volume, 3% to 8% by volume, 3% to 7% by volume, 3% to 6% by volume, 4% to 10% by volume, 4% to 9% by volume, 4% to 8% by volume, 4% to 7% by volume, 4% to 6% by volume, 4% to 5% by volume, 5% to 10% by volume, 5% to 9% by volume, 5% to 8% by volume, 6% to 10% by volume, 6% to 9% by volume, 6% to 8% by volume, 6% to 7% by volume, or the like, preferably 5% to 10% by volume, 5% to 8% by volume, for example, 6% to 10% by volume, 6% to 8% by volume, 6% to 7% by volume, or the like can be inhaled or sucked by subjects for, for example, one to three hours or longer per day and can be continued for, for example, one to three months or longer, four to seven months or longer, one to three years or longer.

Alternatively, in the method of the present invention, for example, the standard concentration for the molecular hydrogen-containing liquid to be administrated to the subject is more than 0 ppm and 1.6 ppm or less. Preferably, the concentration is 2.0 to 5.0 ppm, 2.0 to 6.0 ppm, 2.0 to 7.0 ppm, or 2.0 to 8.0 ppm, or 2 to 9 ppm. More preferably, the concentration is 3.0 to 7.0 ppm, 3.0 to 8.0 ppm, 3.0 to 9.0 ppm, 3.0 to 10 ppm, 4.0 to 7.0 ppm, 4.0 to 8.0 ppm, 4.0 to 9.0 ppm, 4.0 to 10 ppm, 5.0 to 7.0 ppm, 5.0 to 8.0 ppm, 5.0 to 9.0 ppm, 5.0 to 10 ppm, 3.0 to 7.0 ppm, 4.0 to 8.0 ppm, 5 to 7.0 ppm, 5.0 to 8.0 ppm, or 5.0 to 9.0 ppm. Still more preferably, the concentration is more than 0.0 ppm and 10 ppm or less, 1.0 to 10 ppm, 1.5 to 10 ppm, 2.0 to 10 ppm, 3.0 to 10 ppm, 4.0 to 10 ppm, 5.0 to 10 ppm, 6.0 to 10 ppm, or 7.0 to 10 ppm. 200 to 500 mL per dose for intravenous administration or, for example, 500 to 1000 mL per dose for oral administration of a liquid containing molecular hydrogen can continue to be administered to subjects for, for example, 0.5 to three months or longer, four to seven months or longer, one to three years or longer.

The method of the present invention may further be used in combination with a therapeutic agent used for prevention or improvement of human osteoporosis, if necessary. Such a combination use is expected to increase levels of maintenance of bone density and/or prevention or improvement of human osteoporosis.

### EXAMPLE

The present invention is explained more specifically with reference to the following example. However, the example is not intended to limit the scope of the present invention. The diagnosis by doctors also involves personal intuitive opinions based on their experiences, rather than is based on only scientific grounds. Thus, the present invention is not always limited by the diagnosis by doctors.

### Example 1

### <Case 1 of Preventing or Improving Human Osteoporosis by Hydrogen Inhalation>

A 77-year-old woman had a compression fracture at two sites of the lower back 7 years before the present patent application, and the bone density measured was 74%. She had a symptom of stooped posture because the pain in lower back worsened when she walked or carried a heavy object. She received rehabilitation for seven months and continued to visit an osteopathic clinic for one year, but the pain in the lower back was not cured. She started receiving Pralia Subcutaneous Injection (denosumab) once every six months, and her bone density exceeded 80% 18 months later. The bone density raised to 86% in February 2020 and slightly decreased to 84% in August 2020. In November 2020, inhalation of a hydrogen gas was started using a hydrogen gas generator Jobs-mini (hydrogen concentration: 1.5% to 2.0% by volume, the amount of hydrogen in terms of hydrogen concentration of 100%: 30 mL/min) manufactured by MiZ Company Limited, and the patient started inhalation at 11:00 p.m. almost every day. For the first several weeks after starting inhalation, she inhaled the hydrogen gas for about two hours. Then, the duration of inhalation was increased, and she continued inhalation during sleep until about 7:30 to 8:00 a.m. in the following morning. When bone density was measured in March 2021, it had raised to 92%. Other effects of hydrogen gas inhalation included the following effects: improvement in the pain at the compression fracture sites in the lower back, improvement in the gait, feeling tired less easily, feeling that the body became light, weeding in the garden becoming easy, and having a good feeling and feeling refreshed when waking up in the morning.

The present invention can prevent or improve human osteoporosis by administering the composition comprising molecular hydrogen.

What is claimed is:

1. A method for preventing or improving human osteoporosis, comprising administering to a subject in need there of a composition comprising an effective amount of molecular hydrogen.

2. The method according to claim 1, wherein the human osteoporosis is primary osteoporosis and/or secondary osteoporosis.

3. The method according to claim 1, wherein the preventing or improving human osteoporosis is maintaining or raising bone density.

4. The method according to claim 1, wherein the composition is a gas composition having a hydrogen concentration of higher than zero (0) and not higher than 18.5% by volume.

5. The method according to claim 1, wherein the composition is a gas composition which is fed to the subject using a hydrogen gas supply apparatus by inhalation of the composition.
